(19) United States
(12) Patent Application Publication
(10) Pub. No.: US 2023/0092795 A1
(43) Pub. Date: Mar. 23, 2023

(54) COMPOSITIONS, METHODS, KITS, AND SYSTEMS RELATING TO CHARGE-NEUTRAL MICROGELS FOR 3D CELL CULTURE AND PRINTING

(71) Applicant: University of Florida Research Foundation, Inc., Gainesville, FL (US)

(72) Inventors: Thomas Ettor Angelini, Gainesville, FL (US); Brent S. Sumerlin, Gainesville, FL (US); Christopher S. O'Bryan, Gainesville, FL (US); Georg Scheutz, Gainesville, FL (US)

(21) Appl. No.: 17/802,686

(22) PCT Filed: Feb. 26, 2021
(86) PCT No.: PCT/US21/19922

§ 371 Date: Aug. 26, 2022

Related U.S. Application Data
(60) Provisional application No. 62/983,056, filed on Feb. 28, 2020.

(51) Int. Cl. C12N 5/00; C12M 1/12; B33Y 70/00

(52) U.S. Cl. CPC C12N 5/0062; C12M 25/18; B33Y 70/00; C12N 2533/30; C12N 2537/10

(57) ABSTRACT

Described herein are compositions, methods, kits, and systems relating to smooth, spherical microgels which can be charge-neutral. The microgels can be made using an emulsification process. In certain aspects, charge-neutral microgels as described herein are suitable for 3D cell culture, use in perfusion bioreactors, and/or 3D printing of cells for 3D cell culture.

### CROSS-REFERENCE TO RELATED APPLICATIONS

This application claims the benefit of and priority to U.S. Provisional Application Ser. No. 62/983,056, having the title “COMPOSITIONS, METHODS, KITS, AND SYSTEMS RELATING TO CHARGE-NEUTRAL MICROGELS FOR 3D CELL CULTURE AND PRINTING”, filed on Feb. 28, 2020, the disclosure of which is incorporated herein by reference in its entirety.

### STATEMENT REGARDING FEDERALLY SPONSORED RESEARCH

This invention was made with government support under Grant No. W911NF-17-3-0003 awarded by the United States Department of Defense, United States Army under a subaward from Advanced Regenerative Manufacturing Institute. The government has certain rights in the invention.

### BACKGROUND

The printing or placement of biological samples (e.g., cells, cell layers, tissues) into a 3D support medium more accurately and reproducibly models cellular morphology, heterogeneity, and genetic profiles seen in vivo compared to conventional 2D culture. Some existing 3D cell culture techniques rely on polymer scaffolds in which cells are seeded and allowed to adhere. Once the cells are adhered to the scaffold, perfusion of growth media can begin. This method has several disadvantages: (1) cell migration is limited or precluded, (2) cell environments are defined by the structure of the polymer scaffold, (3) the experimental setup is not time-effective, and (4) does not include optical access for microscopy. In addition, cell viability for existing 3D culture methods is generally limited to several days; the passive 3D support medium cannot efficiently expel cellular waste, leading to localized cytotoxic environments and subsequent cell death.

Accordingly, there is a need to address the aforementioned deficiencies and inadequacies, in particular compositions for 3D growth media that can lead to improved perfusion of the 3D growth media.

### SUMMARY

Embodiments of the present disclosure provide for compositions, methods, kits, and systems relating to charge-neutral microgels for 3D cell culture and printing and the like.

An embodiment of the present disclosure includes a three-dimensional (3D) cell culture medium. The 3D cell culture medium can include a plurality of charge-neutral microgel particles and a liquid cell culture medium. The charge-neutral microgel particles are substantially spherical can have a radius of from 0.5 μm to 100 μm.

An embodiment of the present disclosure also includes a kit. The kit can include a 3D cell culture gel having a plurality of charge-neutral microgel particles as above and cell culture media.

An embodiment of the present disclosure also includes a 3D cell culture medium as above and a bioreactor.

An embodiment of the present disclosure also includes method of 3D printing, including depositing one or more cells into a 3D cell culture medium as above, where the 3D cell culture medium is contained in a bioreactor by a 3D printing apparatus.

Other compositions, apparatus, methods, features, and advantages will be or become apparent to one with skill in the art upon examination of the following drawings and detailed description. It is intended that all such additional compositions, apparatus, methods, features and advantages be included within this description, be within the scope of the present disclosure, and be protected by the accompanying claims.

### BRIEF DESCRIPTION OF THE DRAWINGS

Many aspects of the disclosed systems and methods can be better understood with reference to the following drawings. The components in the drawings are not necessarily to scale, emphasis instead being placed upon clearly illustrating the relevant principles. Moreover, in the drawings, like reference numerals designate corresponding parts throughout the several views.

FIG. 1 shows a synthetic scheme for charge-neutral microgels according to the present disclosure. According to the present example is a synthetic scheme for the preparation of uncharged poly(ethylene glycol) (PEG) microgels through an inverse emulsion polymerization containing poly(ethylene glycol) methyl ether acrylate (PEGa) and poly(ethylene glycol) diacrylate (PEGda).

FIG. 2 is an example of a printing apparatus that can be used in conjunction with systems as described herein.

FIG. 3A shows a schematic of precipitation polymerization of charged copolymer microgels; micrographs of microgels were collected to confirm proper synthesis and estimate average particle size.

FIG. 3B shows a schematic of inverse emulsion polymerization of uncharged microgels according to embodiments of the present disclosure; micrographs of microgels were collected to confirm proper synthesis and estimate average particle size. These particles were found to be nearly perfect spheres, in contrast to microgels synthesized by precipitation polymerization.

FIG. 4 shows chemical structures of charged monomers and a schematic of synthesis of charged PEG microgels.

FIG. 5 provides live dead assays demonstrating that the spherical microgels synthesized using inverse emulsion are significantly less toxic than those synthesized using precipitation polymerization. Charge-neutral PEG microgels performed better than charged microgels.

FIG. 6 shows results of perfusion experiments comparing the microgels of the present disclosure to other microgels.

FIG. 7 provides examples of 3D printing of tissues using spherical microgels according to embodiments of the present disclosure.

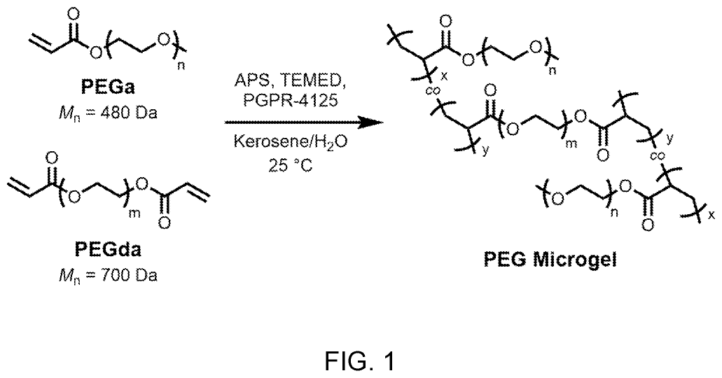

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ at least, unless otherwise indicated, techniques of material science, physics, chemistry, tissue culture, 3D printing, and the like.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is in atmosphere. Standard temperature and pressure are defined as 25° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### Definitions

Unless otherwise defined, all technical and scientific terms used herein have the same meaning as commonly understood by one of ordinary skill in the art of material science, physics, chemistry, tissue culture, 3D printing, and the like. Although methods and materials similar or equivalent to those described herein can be used in the practice or testing of the present disclosure, suitable methods and materials are described herein.

As used in the specification and the appended claims, the singular forms “a,” “an,” and “the” may include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, “charge-neutral” means that there is no net positive negative or positive electrical charge, in particular on the surface of a microgel or microgel composition as described herein. In certain aspects, each microgel of a plurality is charge-neutral.

As used herein, “microgel” refers to colloidal gel particles that are comprised of chemically cross-linked three-dimensional polymer networks. Microgel particles as described herein are spherical, or substantially spherical in shape. “Substantially” spherical refers to a degree of deviation from perfectly spherical that is sufficiently small so as to not measurably detract from the identified property or circumstance. The exact degree of deviation allowable may in some cases depend on the specific context.

### DISCUSSION

Described herein are microgel compositions, methods of synthesis, methods of use, and systems comprising such. In certain aspects, described herein are microgel-based 3D printing and cell culture medium technologies. Described herein is a charge-neutral formulation which is superior to previous formulations, for example liquid-like solids or culture medium comprising swollen hydrogel particles in medium, in many ways.

Without intending to be limiting, for example, advantages of the materials, methods, and systems as described herein include that since the polymers described herein constituting the microgels are charge neutral, meaning the swelling and packing of the microgels is insensitive to changes in solvent composition. Additionally, with current approaches as described herein, the microgels can be synthesized as nearly perfect spheres, which create well-defined pore-spaces between microgels, enabling the material to be perfused much more easily. Materials as described herein are approximately 10,000 times more permeable than previously described materials. In some embodiments, the pore space formed between adjacent charge-neutral microgel particles can be from 50 nm to 10 μm, or approximately 10% of the particle size. The microparticles can have a have a surface roughness of from 0 to 5 micrometers, relative to a perfect spherical surface.

Microqels

Described herein are three-dimensional (3D) cell culture medium containing 1) microgels (also referred to herein as microparticles) that are produced by emulsion polymerization and 2) a liquid cell culture medium.

As disclosed herein, emulsion polymerization results in spherical microparticles comprising a plurality of crosslinked polymers with superior properties for 3D cell culture. Spherical microparticles as described herein are substantially spherical with a constant or near-constant radius at any angle and are not irregularly shaped.

In some embodiments of present disclosure, the disclosed microgels have an average diameter of from about 0.5 μm to 100 μm, including between about 2.5 μm and 32.5 μm in diameter, about 10 μm in diameter, or about 5 μm in diameter.

Packed spherical charge-neutral microgels as described herein have a large pore space, improving permeability about 10,000 times that of existing materials.

In some embodiments, the microgels are charge-neutral. Charge-neutral microgels have a net zero charge on the spherical microparticle surface, which allows the microgels to be insensitive to certain types of cell media and salt concentrations.

In embodiments according to the present disclosure, charge-neutral microgels are comprised of monomers or polymers. In some embodiments, the monomers or polymers include poly(ethylene glycol) methyl ether acrylate (PEGa), poly(ethylene glycol) diacrylate (PEGda), or combinations thereof.

In other embodiments, the monomers or polymers can include acrylamides, N-alkylacrylamides, N,N-dialkylacrylamides, and acrylates. Non-limiting examples include acrylamide (AAm), N,N-dimethylacrylamide, N-isopropylacrylamide, poly(ethylene glycol) acrylate, poly(ethylene glycol) methacrylate, N-vinylcaprolactam, vinyl acetate, 2-hydroxyethyl acrylate, PEG-acrylamide, oligo(ethylene glycol) acrylates, and N-vinylpyrrolidone.

Methods of Synthesis

Described herein are methods of synthesizing charge-neutral microgels.

Methods of synthesizing charge-neutral microgels as described herein comprise synthesizing charge-neutral microgels through an inverse emulsion reaction. In certain aspects, the method comprises: crosslinking emulsified aqueous droplets, wherein emulsified aqueous droplets comprise monomers, polymers, or a combination thereof; emulsifying the aqueous emulsion droplets in a continuous organic phase, wherein the continuous organic phase is prepared by mixing a first organic solvent with a surfactant; preparing an aqueous phase comprising polymers, an aqueous solvent, and an oxidizing agent; mixing and homogenizing the organic and aqueous phases for a period of time in a homogenizer; cooling the mixture and purging with an inert gas; crosslinking the polymers in the mixture with a crosslinking agent; settling the cross-linked microparticles; decanting the first organic solvent; chilling and washing the microparticles; centrifuging the mixture; collapsing the microparticles with a second organic solvent; isolating the collapsed microparticles; and drying the isolated microparticles.

In embodiments, the aqueous emulsion droplets comprise poly(ethylene glycol) methyl ether acrylate (PEGa) and poly(ethylene glycol) diacrylate (PEGda). In embodiments, the PEGa and PEGda can have a ratio of from about 0:100 to about 99:1. In some embodiments, the ratio of PEGa to PEGda can be about 80:20 or about 99:1. In other embodiments, PEGda alone can be used.

In embodiments, the continuous organic phase can be prepared by mixing an organic solvent with a surfactant for a period of time with a stirring means (for example a magnetic stir bar). In some embodiments, the solvent can be kerosene or an n-alkane (e.g. pentane, hexane, hexanes, cyclohexane, heptane, octane). In an embodiment, the organic solvent is kerosene and the surfactant is PGPR-4125.

In embodiments, the aqueous phase comprises a mixture of a 50% stock of PEGa, 25% stock of PEGda, and an oxidant. In an embodiment, the oxidant is ammonium persulfate (APS). In an embodiment, the oxidant is a 10% stock of APS. The aqueous phase is prepared in an aqueous solvent. In an embodiment, the aqueous solvent is water.

In an embodiment, the cooling and purging comprises cooling the mixture in an ice bath and purging with nitrogen gas.

In an embodiment, a crosslinking agent can be included with the polymer. In embodiments, the crosslinker can be such as N,N-methylenebis(acrylamide) (MBA), diethylene glycol diacrylate, pentaerythritol triallyl ether, and N,N-ethylenebis(methacrylamide).

In an embodiment, microparticles are washed with chilled methanol. The wash step can be repeated two to three times.

In an embodiment, to collapse the methanol-swollen microgel particles, they can be added dropwise to a second solvent organic solvent (which can be chilled). The second organic solvent can be diethyl ether or hexanes.

In an embodiment, drying the charge-neutral microparticles can comprise placing in a desiccator and using a pull vacuum.

3D Cell Culture Medium

The 3D cell culture gel described herein may allow for growing diverse cellular structures, including, but not limited to, spheroids, embryoid bodies, tumors, cysts, and microtissues, and may also be used to preserve the structure of cell-laden engineered tissue constructs. In some embodiments, 3D cell culture gel may be dispersed in a liquid cell culture medium to form a 3D cell culture medium (also referred to as a 3D cell growth medium).

Any suitable liquid cell culture medium may be used. A particular liquid cell culture medium may be chosen depending on the types of cells which are to be placed within the 3D cell growth medium. Suitable liquid cell culture media may be human cell growth media, murine cell growth media, bovine cell growth media or any other suitable cell growth media. Depending on the particular embodiment, microgel particles and the liquid cell culture medium may be combined in any suitable combination. For example, in some embodiments, a 3D cell growth medium comprises approximately 0.5% to 10% microgel particles by weight. Some non-limiting examples of cell culture media include Dulbecco's Modified Eagle Medium (DMEM), endothelial cell growth medium (EGM), endothelial basal medium (EBM), William's E medium, or serums such as fetal bovine serum or fetal calf serum, for example.

Furthermore, a 3D cell culture medium may have any suitable combination of mechanical properties, and in some embodiments, the mechanical properties may be tuned via the relative concentration of microgel particles and the liquid cell culture medium. For example, a higher concentration of microgel particles may result in a 3D cell growth medium having a higher elastic modulus and/or a higher yield stress.

The disclosed tunability may be advantageous for controlling the environment around a group of cells placed in a 3D cell growth medium. For example, a 3D cell growth medium may have mechanical properties which are tuned to be similar to those found in vivo so that the cells 3D cell growth medium may emulate the natural environment of the cells. However it should be understood that the mechanical properties of a 3D cell growth medium may not be similar to those found in vivo, or may be tuned to any suitable values, as the disclosure is not so limited.

The microgel particles may be mixed with the liquid cell culture medium using a centrifugal mixer, a shaker, or any other suitable mixing device. During mixing, the microgel particles may swell with the liquid cell culture medium to form a material which is substantially solid when an applied shear stress is below a yield stress. After mixing, entrained air or gas bubbles introduced during the mixing process may be removed via centrifugation, agitation, or any other suitable method to remove bubbles from the 3D cell growth medium.

As used herein, the term “microgel particle” refers to particles suitable for use in a hydrogel, and applies to the particle both when incorporated into a hydrogel and prior to or after incorporated into a hydrogel.

Hydrogels, such as a 3D cell growth medium, may be prepared by adding microgel particles to aqueous solutions at varying concentrations. In some embodiments the aqueous solution may comprise a liquid cell culture medium. As an example, polymer concentrations of less than 10 wt % (with respect to aqueous solution) may be used. Preferably, polymer concentrations of less than 5 wt % are used. Most preferably, polymer concentrations of less than 2 wt % are used. Advantageously, the charge-neutral microgels described herein are almost entirely insensitive to changes in pH.

In one non-limiting example, a 3D cell growth medium comprises approximately 1% to about 10% by mass microgel particles. The microgel particles may be mixed with and swell with any suitable liquid cell growth medium, as described above, to form a 3D cell growth medium which comprises approximately 99.3% to about 99.8% by mass liquid cell culture medium.

When mixed with liquid cell culture medium, the microgel particles may swell with the liquid cell culture medium to form a granular hydrogel material that serves as a 3D cell growth medium. Depending on the particular embodiment, the swollen microgel particles may have a characteristic size at the micron or submicron scales. For example, in some embodiments, the swollen microgel particles may have a size between about 0.5 μm and 100 μm, between about 1 μm and 50 μm, or between about 2.5 μm and 32.5 μm. Other values are also possible.

Properties of Microgel Particles in a Hydrogel

The elastic and shear moduli of the hydrogel is measured by performing an oscillatory frequency sweep at 1% strain across a wide range of frequencies. The elastic shear modulus of the hydrogel is preferred to dominate the viscous shear modulus with a relatively constant value of less than 1000 Pa. In some cases, the elastic shear modulus of the hydrogel is less than 50 Pa. In some cases, the elastic shear modulus of the hydrogel is about 1 Pa to about 1000 Pa, about 10 Pa to about 80 Pa, about 10 Pa to about 60 Pa, about 10 Pa to about 40 Pa, or about 10 Pa to about 20 Pa.

The yield stress of the hydrogel system (e.g., 3D cell growth medium) corresponds to the shear rate independent stress value. The yield stress of the hydrogel is measured by applying a unidirectional shear rate to the hydrogel sample, recording the resulting shear stress and fitting a Hershel-Bulkley model to the resulting stress vs versus strain rate curve. The yield stress of the 3D cell growth medium can be less than 100 Pa. In some cases, the yield stress of the 3D cell growth medium is less than 5 Pa. In some cases, the yield stress of the 3D cell growth medium is about 5 Pa. In some cases, the yield stress of the 3D cell growth medium is about 0.1 Pa to about 100 Pa, about 0.1 Pa to about 50 Pa, about 1 Pa to about 6 Pa, about 1 Pa to about 4 Pa, or about 1 Pa to about 2 Pa.

According to some embodiments, a 3D cell growth medium may be made from materials such that the granular gel material undergoes a temporary phase change due to an applied stress (e.g. a thixotropic or “yield stress” material). Such materials may be solids or in some other phase in which they retain their shape under applied stresses at levels below their yield stress. At applied stresses exceeding the yield stress, these materials may become fluids or in some other more malleable phase in which they may alter their shape. When the applied stress is removed, yield stress materials may become solid again. Stress may be applied to such materials in any suitable way. For example, energy may be added to such materials to create a phase change. The energy may be in any suitable form, including mechanical, electrical, radiant, or photonic, etc.

The terms “yield stress” and “yield stress material” have been used and characterized in different ways in the art. For ease of description herein, the terms “yield stress” and “yield stress material” are used but, unless indicated otherwise, should be understood to be a Herschel-Bulkley yield stress determined using the Herschel-Bulkley equation

σ=σ_{y}+k\dot{γ}^{p}

where σ_{y} is yield stress, σ is shear stress, k is viscosity index of the material, \dot{γ} is shear rate, and p is a positive number, and a material having such a yield stress.

In addition, “yield stress” (i.e., Herschel-Bulkley yield stress) has been measured in different ways in the art. Unless indicated otherwise herein, a yield stress of a sample is determined by shearing the sample in a rheometer using plate-plate geometry and via the Herschel-Bulkley equation, via the following process. Prior to shearing, the rheometer tool surfaces may be roughened to prevent or mitigate slipping at the sample-tool interface. Using the rheometer, the sample is sheared at a variety of shear rates, extending from high shear rates (e.g., 1000 s⁻¹) to low shear rates (0.001 s⁻¹). For each shear rate, the sample is sheared for 30 seconds, after which shear stress data is collected and averaged. A series of shear stress measurements are collected sequentially for each shear rate. These shear rates are then used, via the Herschel-Bulkley equation, to determine (1) whether the material has a yield stress (i.e., a Herschel-Bulkley yield stress), and (2) the yield stress for the material. Those skilled in the art will appreciate that, for a material having a yield stress, a plot of shear stress versus shear rate will exhibit a plateau region at low shear rates, with the data points asymptotically approaching the material's yield stress at low shear rates. The yield stress is the shear stress at these low, near-zero shear rates, or an estimate of shear stress at zero strain rate determined using a low or near-zero shear rate, such as a shear rate of 10⁻³ s⁻¹. As used herein (unless indicated otherwise), a “yield stress material” will be a material that has a yield stress determinable via this process. Those skilled in the art will appreciate that for a yield stress material (i.e., a Herschel-Bulkley yield stress material) at low shear (e.g., a near-zero shear rate), a shear stress is independent of shear rate and instead exhibits only a shear stress dependent only on an elastic component of the material.

A 3D cell growth medium made from a yield stress material, as described above, may enable facile placement and/or retrieval of a group cells at any desired location within the 3D cell growth medium. For example, placement of cells may be achieved by causing a solid to liquid phase change at a desired location in a region of yield stress material such that the yield stress material will flow and be displaced when cells are injected or otherwise placed at the desired location. After injection, the yield stress material may solidify around the placed cells, and therefore trap the cells at the desired location.

However, it should be appreciated that any suitable techniques may be used to deposit cells or other biological materials within the 3D cell growth medium. For example, using a syringe, pipette or other suitable tool, cells may be injected into one or more locations in the 3-D growth medium. In some embodiments, the injected cells may be shaped as a pellet, such as by centrifugation. However, it should be appreciated that a 3D cell growth medium as described herein enables injection of cells suspended in a liquid, which may avoid a centrifugation step in conducting tests.

Regardless of how cells are placed in the medium, the yield stress of the yield stress material may be large enough to prevent yielding due to gravitational and/or diffusional forces exerted by the cells such that the position of the cells within the 3D cell growth medium may remain substantially constant over time. Since the cells are fixed in place, they may be retrieved from the same location at a later time for assaying or testing by causing a phase change in the yield stress material and removing the cells. As described in more detail below, placement and/or retrieval of groups of cells may be done manually or automatically.

A yield stress material as described herein may have any suitable mechanical properties. For example, in some embodiments, a yield stress material may have an elastic modulus between approximately 1 Pa and 1000 Pa when in a solid phase or other phase in which the material retains its shape under applied stresses at levels below the yield stress. In some embodiments, the yield stress required to transform a yield stress material to a fluid-like phase may be between approximately 0.1 Pa and 100 Pa. When transformed to a fluid-like phase, a yield stress material may have a viscosity between approximately 1 Pa s and 10,000 Pa s. However, it should be understood that other values for the elastic modulus, yield stress, and/or viscosity of a yield stress material are also possible, as the present disclosure is not so limited.

In some embodiments, the yield stress may be tuned to match the compressive stress experienced by cell groups in vivo, as described above. Without wishing to be bound by any particular theory, a yield stress material which yields at a well-defined stress value may allow indefinite and/or unrestricted growth or expansion of a group of cells. Specifically, as the group of cells grows, it may exert a hydrostatic pressure on the surrounding yield stress material; this hydrostatic stress may be sufficient to cause yielding of the yield stress material, thereby permitting expansion of the group of cells. In such embodiments, the yielding of the yield stress material during growth of a group of cells may result in the yield stress material maintaining a constant pressure on the group of cells during growth. Moreover, because a yield stress material will yield when an applied stress exceeds the yield stress, a 3D cell growth medium made from a yield stress material may not be able to apply a stress to a group of cells which exceeds the yield stress. The inventors have recognized and appreciated that such an upper bound on the stress applied to a group of cells may help to ensure that cells are not unnaturally constrained, damaged or otherwise altered due to the application of large compressive stresses.

According to some embodiments, a 3D cell growth medium made from a yield stress material may yield to accommodate excretions such as fluids or other extracellular materials from a group of cells disposed within the 3D cell growth medium. Without wishing to be bound by any particular theory, excretion of fluids or other materials from a group of cells may result in an increase in the pressure in the extracellular space; if the pressure exceeds the yield stress of the 3D cell growth medium, the 3D cell growth medium may yield to accommodate the excretions, and a group of cells may excrete fluids or other materials without restriction. Such an ability of a 3D cell growth medium to accommodate cellular excretion may allow the 3D cell growth medium to more closely match an in vivo environment. Moreover, the inventors have recognized and appreciated that a 3D cell growth medium made from a yield stress material may allow for facile removal of cellular excretions for assaying, testing, or any other suitable purpose, as described in more detail below.

A group of cells may be placed in a 3D cell growth medium made from a yield stress material via any suitable method. For example, in some embodiments, cells may be injected or otherwise placed at a particular location within the 3D cell growth medium with a syringe, pipette, or other suitable placement or injection device. In some embodiments an array of automated cell dispensers may be used to inject multiple cell samples into a container of 3-D growth medium. Movement of the tip of a placement device through the 3D cell growth medium may impart a sufficient amount of energy into a region around the tip to cause yielding such that the placement tool may be easily moved to any location within the 3D cell growth medium. In some instances, a pressure applied by a placement tool to deposit a group of cells within the 3D cell growth medium may also be sufficient to cause yielding such that the 3D cell growth medium flows to accommodate the group of cells. Movement of a placement tool may be performed manually (e.g. “by hand”), or may performed by a machine or any other suitable mechanism.

In some embodiments, multiple independent groups of cells may be placed within a single volume of a 3D cell growth medium. For example, a volume of 3D cell growth medium may be large enough to accommodate at least 2, at least 5, at least 10, at least 20, at least 50, at least 100, at least 1000, or any other suitable number of independent groups of cells. Alternatively, a volume of 3D cell growth medium may only have one group of cells. Furthermore, it should be understood that a group of cells may comprise any suitable number of cells, and that the cells may of one or more different types.

Depending on the particular embodiment, groups of cells may be placed within a 3D cell growth medium according to any suitable shape, geometry, and/or pattern. For example, independent groups of cells may be deposited as spheroids, and the spheroids may be arranged on a 3D grid, or any other suitable 3D pattern. The independent spheroids may all comprise approximately the same number of cells and be approximately the same size, or alternatively different spheroids may have different numbers of cells and different sizes. In some embodiments, cells may be arranged in shapes such as embryoid or organoid bodies, tubes, cylinders, toroids, hierarchically branched vessel networks, high aspect ratio objects, thin closed shells, or other complex shapes which may correspond to geometries of tissues, vessels or other biological structures.

Kits

Described herein are kits for 3D cell culture, in particular 3D cell culture for 3D printing or use with 3D printing. Kits as described herein can comprise the 3D cell culture gel (e.g. charge-neutral microgel particles as described herein) and cell culture media. Kits as described herein can further comprise an antibiotic. Some non-limiting examples of antibiotics include Penicillin-streptoymycin, gentamicin, aminoglycosides or any antimicrobial peptides.

Systems

Systems as described herein can comprise charge-neutral microparticle compositions and/or kits as described herein. Systems as described herein can further comprise bioreactors, in which a 3D cell growth media can be contained, the 3D cell growth media comprising microparticles as described herein. The bioreactors can be perfusion-enabled bioreactors. Systems can further comprise one or more 3D printing apparatuses for printing one or more cells into compositions comprising charge-neutral microparticles as described herein.

Additional aspects of bioreactors that are suitable for use with the embodiments of systems as described herein, in particular perfusion-enabled bioreactors, can be found in PCT/US2019/017316, filed on Feb. 8, 2019, and published as WO 2019/157356 A1 on Aug. 15, 2019, which is incorporated by reference herein in its entirety.

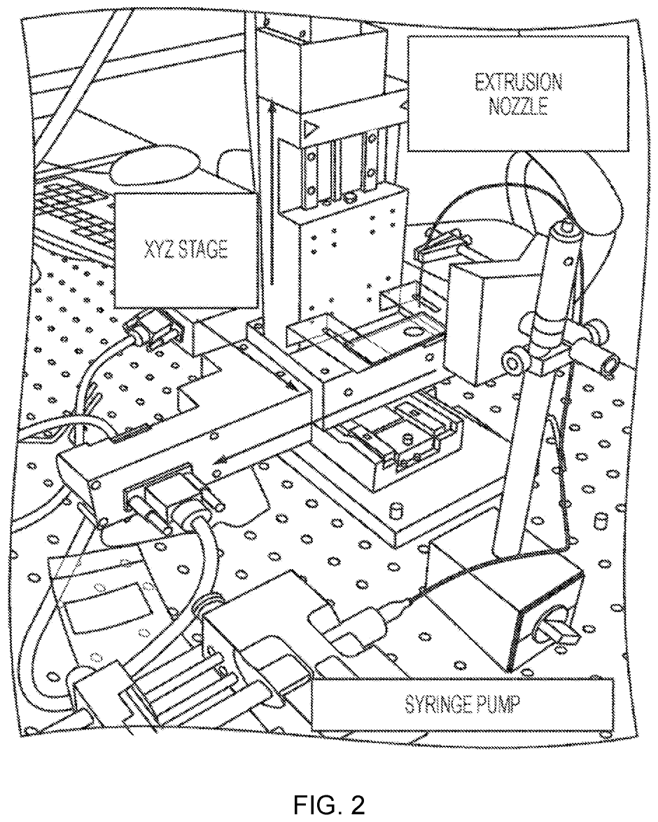

Embodiments of printing apparatuses can be found in PCT/US16/17810, filed on Feb. 12, 2016, and PCT/US2016/052102, filed on Sep. 16, 2016, both of which are incorporated by reference herein in its entirety. FIG. 2 shows an example of a printing apparatus that can be used.

Methods of Use

Also described herein are methods of using charge-neutral microparticles as described herein. Methods of using charge-neutral microparticles can comprise forming a 3D cell growth media by mixing cell culture media and charge-neutral microparticles as described herein. The 3D cell growth media can be placed or otherwise contained in bioreactors as described herein. Additionally, a printing apparatus (for example a 3D printing apparatus as described herein), can be used to print one or more cells into charge-neutral microparticle compositions as described herein.

According to some embodiments, a 3D cell growth medium including charge-neutral microparticles may enable 3D printing of cells to form a desired pattern in three dimensions. For example, a computer-controlled injector tip may trace out a spatial path within a 3D cell growth medium and inject cells at locations along the path to form a desired 3D pattern or shape. Movement of the injector tip through the 3D cell growth medium may impart sufficient mechanical energy to cause yielding in a region around the injector tip to allow the injector tip to easily move through the 3D cell growth medium, and also to accommodate injection of cells. After injection, the 3D cell growth medium may transform back into a solid-like phase to support the printed cells and maintain the printed geometry. However, it should be understood that 3D printing techniques are not required to use a 3D cell growth medium as described herein.

A 3D cell growth medium including charge-neutral microparticles may also allow for facile retrieval of groups of cells from within the cell growth medium via a reversal of the steps used to deposit the cells. For example, cells may be removed by simply moving a tip of a removal device such as a syringe or pipette to a location where a group of cells is disposed, and applying suction to draw the cells from the cell growth medium. As described above, movement of the tip of the removal device through the 3D cell growth medium may impart sufficient energy to the material to cause yielding and accommodate removal of the cells from the 3D cell growth medium. Such an approach may be used, for example, as part of a test process in which multiple cell samples are deposited in 3D cell growth medium. Those deposited cells may be cultured under the same conditions, but different ones of the samples may be exposed to different drugs or other treatment conditions. One or more samples may be harvested at different times to test impact of the treatment conditions on the cells.

In some embodiments, a 3D cell growth medium may be used to support and/or preserve the structure of a cell-laden engineered tissue construct. For example, a tissue construct including a scaffold or other suitable structure on which a plurality of cells is disposed may be placed into a 3D cell growth medium. The 3D cell growth medium may provide support to preserve a complex structure of the tissue construct while also providing a 3D environment for cell growth which may mimic that found in vivo.

It should be appreciated that one or more compounds may be deposited in conjunction with and/or adjacent to cells. For example, soluble, non-cellular components could be deposited in conjunction with the cells. These might include structural proteins (e.g. collagens, laminins), signaling molecules (growth factors, cytokines, chemokines, peptides), chemical compounds (pharmacologic agents), nucleic acids (e.g. DNA, RNAs), and others (nano-particles, viruses, vectors for gene transfer).

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1

1.1. Methods:

Uncharged (i.e. charge-neutral) poly(ethylene glycol) (PEG) microgels are synthesized through an inverse emulsion reaction; aqueous emulsion droplets containing poly(ethylene glycol) methyl ether acrylate (PEGa) and poly(ethylene glycol) diacrylate (PEGda) are emulsified in a continuous organic phase. The organic phase is prepared by mixing 500 mL of kerosene (Sigma) with 3.5 g of PGPR-4125 surfactant (Palsgaard) with a stir bar in a 1 L beaker for 20 min. Separately, the aqueous phase is prepared containing measured amounts of PEGa, PEGda, APS and water (Table 1). The aqueous and organic phases are mixed in a 2 L beaker placed in an ice bath and subsequently homogenized for 5 min at 8000 RPM (homogenizer). The mixture is transferred to a 1 L round bottom flask submerged in an ice bath and purged with nitrogen for 1 h under continuous stirring. After purging, the flask is removed from the ice bath and 3 mL of TEMED (Sigma) is added under continuous mixing for 4 hr.

TABLE 1
Polymer concentrations for PEG microgels

| Polymer | PEGa | PEGda | APS | |
|---|---|---|---|---|
| Concentration | 50% | 25% | 10% | |
| (wt %) | stock | stock | stock | Water |
| 25 wt % | 73.91 | 2.18 | 2.25 | 71.66 |
| 20 wt % | 59.13 | 1.74 | 2.25 | 86.88 |
| 15 wt % | 44.35 | 1.31 | 2.25 | 102.10 |

After crosslinking has completed, the microparticles are allowed to settle to the bottom of the flask and the kerosene is decanted. The microparticles are subsequently washed chilled methanol to remove the remaining kerosene and surfactant. The particles are washed by adding dropwise to 1.5 L of chilled methanol under continuous mixing and stirring for an additional 2 h before letting the particles sediment overnight. The particles are then decanted the cleaning repeated with an additional 1-1.5 L of chilled methanol, followed by allowing the microparticles to sediment overnight. After decanting the excess methanol, the remaining microparticles are centrifuged down. To collapse the microparticles, the methanol swollen microgels are added dropwise to 1 L of freezer-chilled diethyl ether (or Hexanes) while continuously stirring. This step may be repeated a second time to get nice, collapsed PEG microgels. After the second wash in ether, the excess ether can be decanted and microgels transferred to clean 50 mL tubes. The tubes are then placed in a desiccator and vacuum pulled for 24 h.

The following non-limiting concentrations of stock solutions can be used in varying proportions with water to form the aqueous droplet phase: 50 wt % PEGa; 25 wt % PEGda; 10 wt % APS. In Table 1 above, the relative proportions of each are given along with the extra water that should be added.

TABLE 2
Supplies and Part Numbers

| | |
|---|---|
| PEGa 480 MW | 454990 (Sigma Aldrich) |
| PEGda 700 MW | 455008 (Sigma Aldrich) |
| APS | A3678 (Sigma Aldrich) |
| TEMED | BP150 (Fisher Scientific) |
| Kerosene | 329460 (Sigma Aldrich) |
| PGPR-4125 | Paalsgaard |
| Methanol | BPA413 20 (Fisher Scientific) |
| Diethyl Ether | 615080040 (Fisher Scientific) |
| Hexanes | BPH291S-4 (Fisher Scientific) |
| 500 mL Centrifuge Tubes | 431123 (Fisher Scientific) |
| 500 mL Centrifuge Tube Cushions | 431124 (Fisher Scientific) |

1.2. Embodiments of Microgels

In some embodiments, the microgels can have from about 7 wt % to about 95 wt % polymer, or from about 10 wt % and about 25 wt % polymer. The polymer can have a PEGa to PEGda ratio of from about 0:100 to about 99:1 or from about 80:20 to about 99:1.

### Example 2

### Characteristics of Neutral Gels Synthesized Through Emulsion Polymerization

Seven different types of microgels having seven different chemical formulations were synthesized and tested for cytotoxicity. Three of these seven types of microgels were made by a precipitation polymerization method and the remaining four were made by an inverse emulsion polymerization method. Tests focused on the potential impact different charged species may have on cell viability, and therefore cationic, anionic, zwitterionic, and uncharged microgels were synthesized. Each of the charged types of microgels were synthesized using both polymerization routes; the neutral microgels were only prepared using the inverse emulsion polymerization route.

The zwitterionic microgels performed nearly as well as PEG in terms of cell viability, but zwitterionic microgels exhibited swelling behavior sensitive to the ionic strength of their solvent. Since exchanging media containing a variety of different compounds is desirable, it follows that a microgel that is both insensitive to solvent composition and causes insignificant levels of cytotoxicity represents the most robust material for such work. Thus, based on the results provided below, the charge-neutral PEG microgels were the best candidate system for 3D printing, culturing, and assaying liver microtissues (e.g. liver tissues).

Results

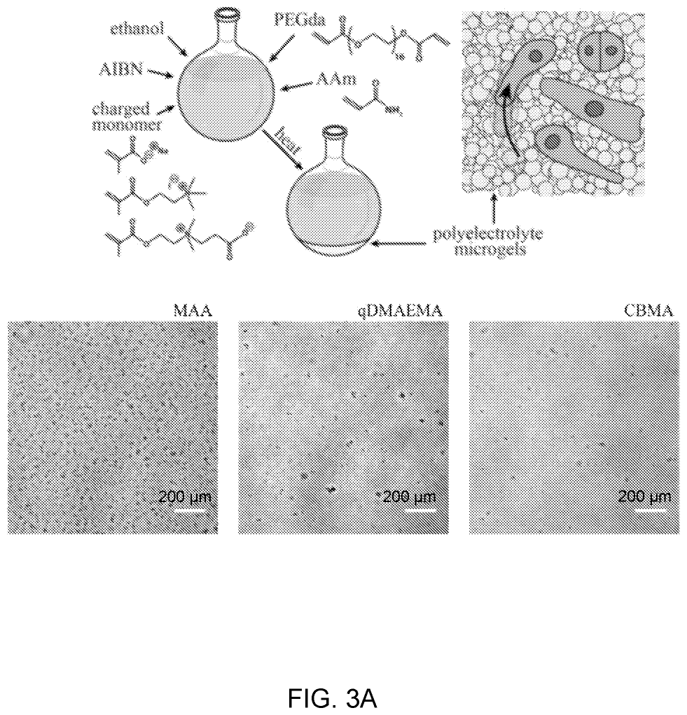

To test the impact charged groups may have on hepatocyte viability, cationic, anioinic, and zwitterionic microgels were synthesized following previously established precipitation polymerization protocols we [C. S. O'Bryan, et. al., ACS Applied Bio Materials, 2 1509 (2019)]. These microgels were made from the neutral polyacrylamide (pAAm) co-monomer, neutral polyethylene glycol diacrylate (PEGda) crosslinker, and additional charged co-monomers. Anionic microgels contained methacrylic acid (MAA); cationic microgels contained quaternized 2-(dimethylamino)ethyl methacrylate (qDMAEMA); zwitterionic microgels contained carboxybetaine methacrylate (CBMA). A schematic of the polymerization scheme is shown in FIG. 3A along with micrographs of the gel particles.

After comparing candidate chemistries for un-charged microgels, formulations entirely made from PEG polymers were chosen. PEG's excellent resistance to fouling and molecular adhesion, as well as its biocompatibility, motivated this choice. The solubility of PEG in organic solvents like ethanol and methanol led to a different polymerization route, which was an inverse emulsion polymerization in which PEG precursors were dispersed within aqueous droplets suspended in an organic liquid phase. Charge neutral microgels were synthesized from polyethylene glycol (PEG). By testing different formulations and synthesis protocols, producing highly spherical microgels with a particle-size distribution that peaked at around 10 μm diameter. FIG. 3B shows micrographs of microgels formed by emulsion polymerization. A detailed synthesis protocol is included later in this example. These particles were found to be nearly perfect spheres, in contrast to microgels synthesized by precipitation polymerization shown in the micrographs in FIG. 3A.

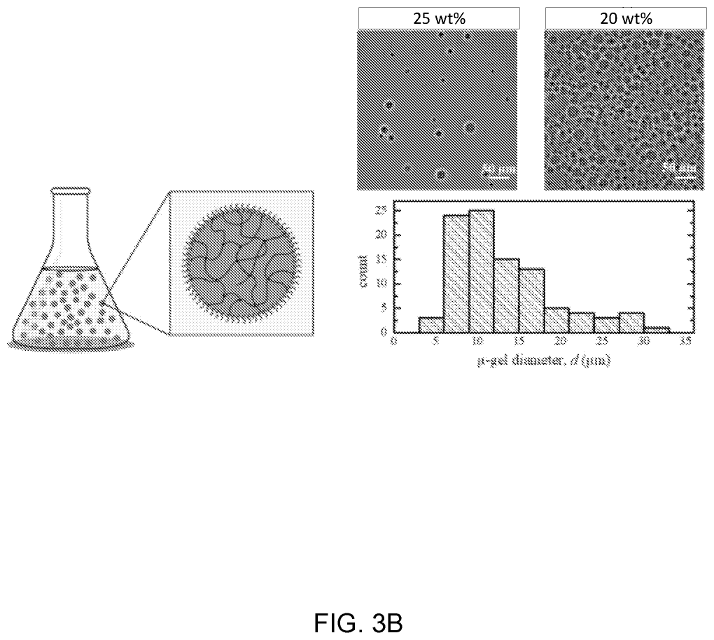

Since charged and uncharged microgels were synthesized using different methods that produced microgels with different shapes, an additional set of charged gels were synthesized using the inverse emulsion protocol. The same charged co-monomers previously used were incorporated into the new PEG microgel formulation. The chemical structures of the charged monomers and fully reacted co-polymers are shown in FIG. 4 and example compositions are detailed later in this example. Since the cationic qDMAEMA co-monomer requires an additional synthesis step, also detailed later in this example.

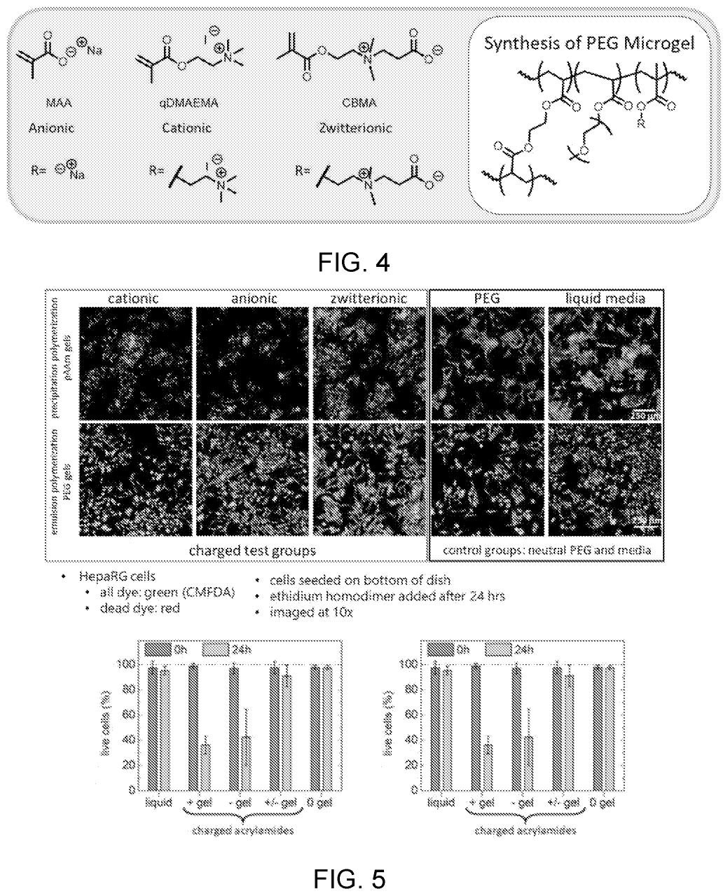

To compare the seven different microgel formulations described above in terms of their suitability for culture of 3D bio-printed liver structures, short-term (0-24 h) cytotoxicity assays were performed and the results compared to control samples in pure liquid media (no microgels). Cytotoxicity was measured using a standard live-dead assay. Cells were plated on a standard culture surface and left to attach and spread for approximately 24 hours. The liquid media was then replaced with microgels swollen in liquid media. The percent live cells were measured between one and three hours after the media exchange (labeled 0 h in FIG. 4) and also after 24 hours. For each of the seven microgel formulations, cell viability was measured on N=3 different samples at three different fields of view within each sample. The mean and standard deviation across measurements for each microgel formulation are shown in FIG. 5. It was found that anionic and cationic microgels from the precipitation polymerization group were very cytotoxic to HepaRG hepatocytes. By contrast, the same chemical formulations made from inverse emulsion polymerization were far less cytotoxic and statistically equivalent to the liquid media control group. For both cases, the zwitterionic gels were the best charged species, performing nearly as well as the control samples. The uncharged PEG microgels performed better in each case than charged microgels (see FIG. 5). That difference in cytotoxicity between microgels prepared by precipitation polymerization and those prepared by inverse emulsion polymerization may arise from microgel shape. Microgels from inverse emulsions are perfectly spherical, while microgels from precipitation polymerization are irregularly shaped. The spherical microgels pack onto the cell surface like a layer of hard spheres, making far fewer direct contact points than the irregularly shaped gels, that more uniformly blanket the cells.

Outcome

The uncharged PEG gels prepared by inverse emulsion polymerization exhibit the lowest levels of cytotoxicity, statistically indistinguishable from liquid culture controls. Moreover, the uncharged gels are expected to be highly insensitive to aqueous solvent composition. However, the uncharged gels benefit from vigorous mechanical agitation to disperse and homogenize in liquid growth media.

Neutral PEG Microgel Synthesis Details and Protocol

The following materials, vendors, and part numbers were used in the study described in this example: PEGa 480 MW (Sigma Aldrich 454990); PEGda 700 MW (Sigma Aldrich 455008); Ammonium Persulfate (Sigma Aldrich A3678); TEMED (Fisher Scientific BP150); PGPR (Paalsgaard 4125); Kerosene (Sigma Aldrich 329460); Methanol (Fisher Scientific BPA413 20); Diethyl Ether (Fisher Scientific 615080040); 500 mL Centrifuge Tubes (Fisher Scientific 431123); 500 mL Centrifuge Tube Cushions (Fisher Scientific 431124); Ice; MilliQ Water. These materials are not intended to be limiting and alternatives may be used as provided for elsewhere in the disclosure, or as can be envisioned by one of ordinary skill in the art. Examples of PEG micro-gel compositions can be found in Table 1 above.

An example synthesis protocol for charge-neutral particles is provided:

1. Create Organic Phase in 1L beaker; a. Add 3.5 g PGPR to the beaker; b. Add 500 mL Kerosene to the beaker; c. Stir for 15 min.

2. Create Aqueous Phase in 200 mL beaker; a. Make 3 g of 10% ammonium persulfate solution; b. Add PEGa to beaker based on desired concentration (see Table 1); c. Add PEGda to beaker based on desired concentration (see Table 1); d. Add 2.25 g ammonium persulfate; e. Add Water based on desired concentration (see Table 1); f. Stir for 5 min.

3. Combine organic and aqueous in 1 L beaker; a. Remove stir bar from organic solution; b. Place beaker containing organic phase on ice; c. Add in aqueous mixture; d. Homogenize for approximately 5 min at 7000 rpm.

4. Purge and stir solution; a. Place solution in round bottom flask; b. Put flask over ice and stir solution; c. Purge with Nitrogen for 1 hour.

5. Reaction of solution with TEMED; a. Remove round bottom flask from ice; b. Raise gas line from solution but still keep in flask to maintain nitrogen atmosphere; c. Add 3 mL TEM ED to the solution; d. Stir for 1 hour; e. Remove seal to allow air back in solution; f. Stir for 30 min.; g. Distribute evenly into eight 500 mL conical tubes.

6. Perform methanol wash of gel; a. Add methanol to make total volume in each conical tube approximately 500 mL; b. Weight tubes to verify total weights are within +1-1% of each other; c. Shake tubes vigorously for approximately 1 minute each; d. Centrifuge at 4000 rpm for 30 minutes; e. Remove supernatant; f. Repeat.

7. Collapse gels in ether; a. Add diethyl ether to tubes to make total volume approximately 250 mL; b. Weight tubes to verify total weights are within +1-1% of each other; c. Shake tubes vigorously for approximately 1 minute each; d. Centrifuge at 4000 rpm for 5 minutes; e. Remove supernatant; f. Repeat.

8. Desiccate gel; a. Distribute solution into approximately 15, 50 mL tubes with approximately 5 mL of gel placed in each; b. Desiccate for approximately 12 hours under vacuum.

The charged poly(ethylene glycol) (PEG) microgels were synthesized through an inverse emulsion reaction; aqueous emulsion droplets containing poly(ethylene glycol) methyl ether acrylate (PEGa), charged comonomer (methacrylic acid (MAA), quaternized 2-(dimethylamino)ethyl methacrylate (qDMAEMA), carboxybetaine methacrylate (CBMA)), and poly(ethylene glycol) diacrylate (PEGda) are emulsified in a continuous organic phase, following the protocol described above with the only modification being the addition of charged co-monomer. The compositions used to synthesize the charged PEG gels are given below in

TABLE 3
The suppliers and part Numbers for charged monomers are as follows: CBMA, M2359
(TCI America); MAA, 155721 (Sigma Aldrich); DMAEMA, 234907-100 ML (Sigma Aldrich).

| | | | | Charged Comonomer | | | |
|---|---|---|---|---|---|---|---|
| Polymer | PEGa | PEGda | APS | 17 mol % incorporation | | | |
| Concentration | 50 wt % | 25 wt % | 10 wt % | MAA | qDMAEMA | CBMA | |
| (wt %) | stock | stock | stock | anionic | cationic | zwitterionic | Water |
| 25 wt % | 73.91 g | 2.18 g | 2.25 g | 1.126 g | 3.904 g | 3.003 g | 71.66 g |

### Example 3

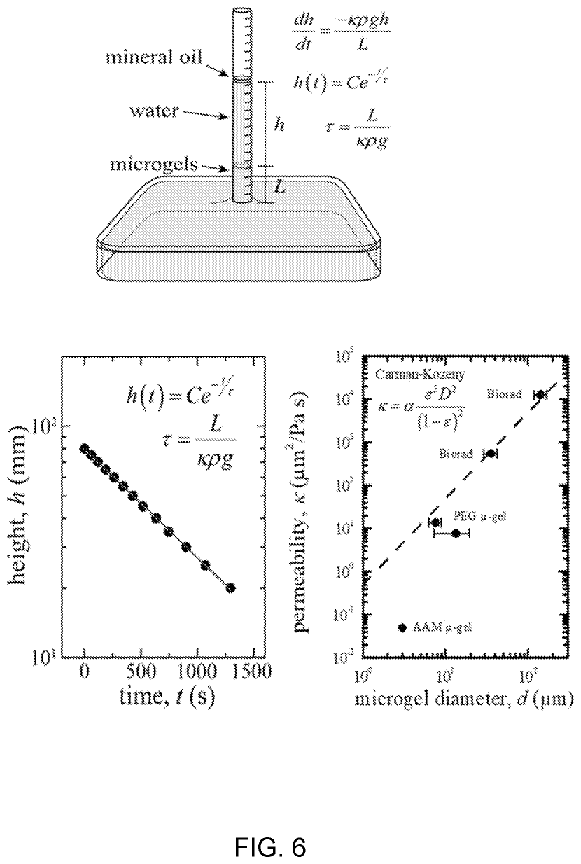

The permeability of charge-neutral packed microgel formed by the emulsification methods described herein was measured in gravity-driven flow columns. The heights of water columns were measured over time and were found to decrease exponentially, as expected (FIG. 6). A permeability was determined from the exponential decay constant. For spherical objects (PEG microgels and Biorad chromatography beads), the Carman-Kozeny formula predicts the relationship between particle dimeter and permeability. The charge-neutral microgels described herein can have a permeability of about 0.1 μm² to 10000 μm².

Nonspherical objects like pAAm microgels deviate strongly from this relationship and pack into far less permeable configurations, as shown by the neutral microgel made using precipitation polymerization, (AAM), in FIG. 6. The primary conclusion from these studies is that spherical PEG microgels are an excellent candidate for a new 3D printing/culture medium because they are highly permeable when compared to non-spherical microgels.

### Example 4

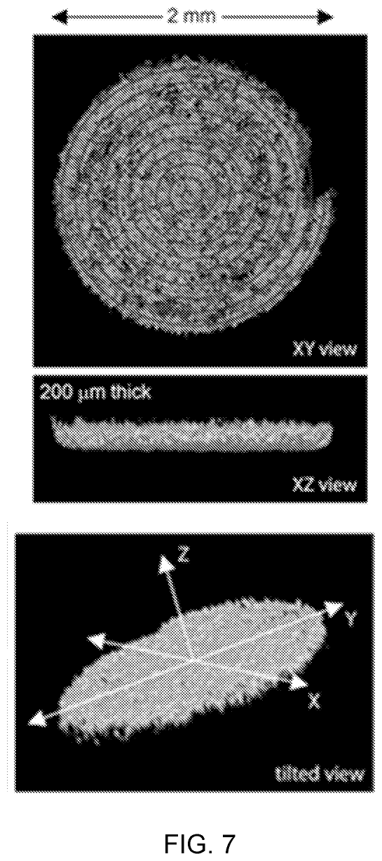

FIG. 7 provides an example of 3D printing into the spherical microgels of the present disclosure. As shown, the biofabricated tissues had a spiral pitch of about 100 μm, and a tissue thickness of about 200 μm. The tissue had a variable diameter. The cells were printed with extracellular matrix (ECM). Tissues were printed in spiral paths to create tissues in a single smooth motion, reducing the appearance of unwanted features. Spiral print paths reduce seams and stagnation points. The feature width is matched to the spiral pitch and the resulting tissue is 200 mm thick. The hepatocytes investigated include immortalized (HepG2), primary (IVAL, ThermoFisher), and stem cell-derived (Takara, HepaRG, in-house iPSC). This strategy has been employed using several different hepatocyte sources.

Aspects of the Disclosure

The present disclosure will be better understood upon reading the following numbered aspects, which should not be confused with the claims. Any of the numbered aspects below can, in some instances, be combined with aspects described elsewhere in this disclosure and such combinations are intended to form part of the disclosure.

Aspect 1. A three-dimensional (3D) cell culture medium comprising: a plurality of charge-neutral microgel particles; and a liquid cell culture medium, wherein the charge-neutral microgel particles are spherical, and wherein the charge-neutral microgel particles have a radius of from 0.5 μm to 100 μm.
Aspect 2. The 3D cell culture medium according to aspect 1, wherein the charge-neutral microgel particles have a surface roughness of from 0 to 5 micrometers, relative to a perfect spherical surface.
Aspect 3. The 3D cell culture medium according to aspects 1 or 2, wherein the 3D cell culture gel comprises from 0.1% to 10% polymer.
Aspect 4. The 3D cell culture medium according to any of aspects 1-3, wherein a pore space formed between adjacent charge-neutral microgel particles is from 50 nm to 10 μm.
Aspect 5. The 3D cell culture medium according to any of aspects 1-4, wherein the charge-neutral microgel particles are formed by crosslinking emulsified aqueous droplets, wherein emulsified aqueous droplets comprise monomers, polymers, or a combination thereof.
Aspect 6. The 3D cell culture medium according to aspect 5, wherein the charge-neutral microgel particles are formed by a process comprising: emulsifying the aqueous emulsion droplets in a continuous organic phase, wherein the continuous organic phase is prepared by mixing a first organic solvent with a surfactant; preparing an aqueous phase comprising polymers, an aqueous solvent, and an oxidizing agent; mixing and homogenizing the organic and aqueous phases for a period of time in a homogenizer; cooling the mixture and purging with an inert gas; crosslinking the polymers in the mixture with an initiator; settling the cross-linked microparticles; decanting the first organic solvent; chilling and washing the microparticles; centrifuging the mixture; collapsing the microparticles with a second organic solvent; isolating the collapsed microparticles; and drying the isolated microparticles.
Aspect 7. The 3D cell culture medium according to aspect 6, wherein the first organic solvent is kerosene.
Aspect 8. The 3D cell culture medium according to aspects 6 or 7, wherein the surfactant is PGPR-4125.
Aspect 9. The 3D cell culture medium according to any one of aspects 6 to 8, wherein the second organic solvent is a diethyl ether or a hexane.
Aspect 10. The 3D cell culture medium according to any one of aspects 6 to 9, wherein the polymers of the aqueous phase comprise a mixture of a 50% stock of PEGa and a 25% stock of PEGda.
Aspect 11. The 3D cell culture medium according to any one of aspects 6 to 10, wherein the oxidizing agent is ammonium persulfate.
Aspect 12. The 3D cell culture medium according to any one of aspects 6 to 11, wherein the initiator is tetramethylethylenediamine (TEMED).
Aspect 13. The 3D cell culture medium according to aspect 5, wherein the aqueous emulsion droplets comprise poly(ethylene glycol) methyl ether acrylate (PEGa) and poly(ethylene glycol) diacrylate (PEGda).
Aspect 14. The 3D cell culture medium according to any of aspects 1-13, wherein the charge-neutral microgel particles comprise a crosslinked polymer network; wherein the crosslinked polymer network comprises poly(ethylene glycol) methyl ether acrylate (PEGa) and poly(ethylene glycol) diacrylate (PEGda); wherein a ratio of PEGa to PEGda is about 80:20; and wherein the 3D cell culture gel comprises about 10 wt % to about 25 wt % crosslinked polymer network.
Aspect 15. The 3D cell culture medium of any one of aspects 1 to 14, wherein the liquid cell culture medium has a permeability of 0.1 μm² to 10000 μm².
Aspect 16. The 3D cell culture medium according to any one of aspects 1 to 15, wherein the 3D cell culture medium has a yield stress such that the cell culture medium undergoes a phase change from a first solid phase to a second liquid phase upon application of a shear stress greater than the yield stress.
Aspect 17. The 3D cell culture medium according to any one of aspects 1 to 16, wherein the yield stress is from 0.1 Pa to 100 Pa.
Aspect 18. The 3D cell culture medium according to any one of aspects 1 to 17, wherein the concentration of microgel particles is from 0.05% to 1.0% by weight.
Aspect 19. A kit, comprising: a 3D cell culture gel comprising a plurality of charge-neutral microgel particles and cell culture media; wherein the charge-neutral microgel particles are substantially spherical, and wherein the charge-neutral microgel particles have a radius of from 5 nm to 500 nm.
Aspect 20. The kit according to aspect 19, further comprising an antibiotic.
Aspect 21. The kit according to any one of aspects 19-20, further comprising one or more bioreactors.
Aspect 22. The kit according to any one of aspects 19-21, wherein the cell culture media is selected from Dulbecco's Modified Eagle Medium (DMEM), endothelial cell growth medium (EGM), endothelial basal medium (EBM), and William's E medium.
Aspect 23. A system, comprising: a 3D cell culture medium according to any one of claims 1 to 18; and a bioreactor.
Aspect 24. The system according to aspect 23, wherein the bioreactor is a perfusion-enabled bioreactor.
Aspect 25. The system according to aspects 23 or 24, further comprising a 3D printing apparatus configured to print into the bioreactor.
Aspect 26. A method of 3D printing, comprising depositing one or more cells into a 3D cell culture medium contained in a bioreactor by a 3D printing apparatus, wherein the 3D cell culture medium comprises the 3D cell culture medium according to any one of claims 1 to 18.

Unless defined otherwise, all technical and scientific terms used have the same meaning as commonly understood by one of ordinary skill in the art to which this disclosure belongs. Although any methods and materials similar or equivalent to those described can also be used in the practice or testing of the present disclosure, the preferred methods and materials are now described.

Embodiments of the present disclosure will employ, unless otherwise indicated, techniques of separating, testing, and constructing materials, which are within the skill of the art. Such techniques are explained fully in the literature. It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims

1. A three-dimensional (3D) cell culture medium comprising:
a plurality of charge-neutral microgel particles; and
a liquid cell culture medium,
wherein the charge-neutral microgel particles are substantially spherical, and
wherein the charge-neutral microgel particles have a radius of from 0.5 μm to 100 μm.

2. The 3D cell culture medium according to claim 1, wherein the charge-neutral microgel particles have a surface roughness of from 0 to 5 micrometers, relative to a perfect spherical surface.

3. The 3D cell culture medium according to claim 1, wherein the 3D cell culture gel comprises from 0.1% to 10% polymer.

4. The 3D cell culture medium according to claim 1, wherein a pore space formed between adjacent charge-neutral microgel particles is from 50 nm to 10 μm.

5. The 3D cell culture medium according to claim 1, wherein the charge-neutral microgel particles are formed by crosslinking emulsified aqueous droplets, wherein emulsified aqueous droplets comprise monomers, polymers, or a combination thereof.

6. The 3D cell culture medium according to claim 5, wherein the charge-neutral microgel particles are formed by a process comprising:
emulsifying the aqueous emulsion droplets in a continuous organic phase, wherein the continuous organic phase is prepared by mixing a first organic solvent with a surfactant;
preparing an aqueous phase comprising polymers, an aqueous solvent, and an oxidizing agent;
mixing and homogenizing the organic and aqueous phases for a period of time in a homogenizer;
cooling the mixture and purging with an inert gas;
crosslinking the polymers in the mixture with an initiator;
settling the cross-linked microparticles;
decanting the first organic solvent;
chilling and washing the microparticles;
centrifuging the mixture;
collapsing the microparticles with a second organic solvent;
isolating the collapsed microparticles;
drying the isolated microparticles.

7. The 3D cell culture medium according to claim 6, wherein the first organic solvent is kerosene.

8. The 3D cell culture medium according to claim 6, wherein the surfactant is PGPR-4125.

9. The 3D cell culture medium according to claim 6, wherein the second organic solvent is a diethyl ether or a hexane.

10. The 3D cell culture medium according to claim 6, wherein the polymers of the aqueous phase comprise a mixture of a 50% stock of PEGa and a 25% stock of PEGda.

11. The 3D cell culture medium according to claim 6, wherein the oxidizing agent is ammonium persulfate.

12. The 3D cell culture medium according to claim 6, wherein the initiator is tetramethylethylenediamine (TEMED).

13. The 3D cell culture medium according to claim 5, wherein the aqueous emulsion droplets comprise poly(ethylene glycol) methyl ether acrylate (PEGa) and poly(ethylene glycol) diacrylate (PEGda).

14. The 3D cell culture medium according to claim 1, wherein the charge-neutral microgel particles comprise a crosslinked polymer network;
wherein the crosslinked polymer network comprises poly(ethylene glycol) methyl ether acrylate (PEGa) and poly(ethylene glycol) diacrylate (PEGda);
wherein a ratio of PEGa to PEGda is about 80:20; and
wherein the 3D cell culture gel comprises about 10 wt % to about 25 wt % crosslinked polymer network.

15. The 3D cell culture medium of claim 1, wherein the liquid cell culture medium has a permeability of 0.1 μm² to 10000 μm^{t}.

16. The 3D cell culture medium according to claim 1, wherein the 3D cell culture medium has a yield stress such that the cell culture medium undergoes a phase change from a first solid phase to a second liquid phase upon application of a shear stress greater than the yield stress; and wherein the yield stress is from 0.1 Pa to 100 Pa.

17. (canceled)

18. The 3D cell culture medium according to claim 1, wherein the concentration of microgel particles is from 0.05% to 1.0% by weight.

19. (canceled)

20. The 3D cell culture medium according to claim 1, further comprising an antibiotic.

21-22. (canceled)

23. A system, comprising:
a 3D cell culture medium comprising:
a plurality of charge-neutral microgel particles, and
a liquid cell culture medium,
wherein the charge-neutral microgel particles are substantially spherical,
wherein the charge-neutral microgel particles have a radius of from 0.5 μm to 100 μm; and
a bioreactor.

24. (canceled)

25. The system according to claim 23, further comprising a 3D printing apparatus configured to print into the bioreactor.

26. (canceled)
